(19) United States
(12) Patent Application Publication
(10) Pub. No.: US 2017/0096661 A1
(43) Pub. Date: Apr. 6, 2017

(54) CONCENTRATING NUCLEIC ACIDS IN URINE

(71) Applicant: TROVAGENE, INC, San Diego, CA (US)

(72) Inventors: Karena Kosco, San Diego, CA (US); Jason Poole, San Diego, CA (US); Mark G. Erlander, San Diego, CA (US)

(73) Assignee: TROVAGENE, INC, San Diego, CA (US)

(21) Appl. No.: 15/298,512

(22) Filed: Oct. 20, 2016

Related U.S. Application Data
(63) Continuation of application No. PCT/US2015/026960, filed on Apr. 21, 2015.
(60) Provisional application No. 61/982,855, filed on Apr. 22, 2014.

(51) Int. Cl. C12N 15/10; B01D 71/68; B01D 71/10

(52) U.S. Cl. CPC C12N 15/1017; B01D 71/10; B01D 71/68

(57) ABSTRACT

Provided is a method for concentrating nucleic acids in urine which can be performed without use of toxic reagents and without centrifugation steps. The method allows a 10× or greater concentration of the nucleic acids, removes impurities, and allows processing of volumes greater than 20 ml as a single sample.

### PRIORITY

This application claims priority under 35 USC §119(e) of U.S. Provisional Application, 61/982,855, filed Apr. 22, 2014, which is hereby incorporated by reference in its entirety.

### FIELD OF THE DISCLOSURE

This disclosure relates to an advance in the preparation of nucleic acid molecules for extraction, isolation, and/or detection or analysis. The disclosure relates to nucleic acid molecules in urine and the concentrating of those molecules.

### BACKGROUND

Historically, urine has not been considered an ideal source of nucleic acids, and especially cell-free or circulating cell-free nucleic acids, due to the low concentration of these molecules in urine. For certain applications (e.g. diagnostics, clinical monitoring, treatment response, etc . . . ), there is a particular and critical need for non-invasive and safe methods of biological sample collection and processing.

A variety of purification strategies have been used for the separation of nucleic acids from urine. These include precipitation, aqueous two-phase separation, and also adsorption using anion-exchange columns. While these methods may be useful for processing small volumes of urine, they are especially cumbersome and labor intensive when greater volumes (e.g. 20 ml or more) per individual sample are needed to be processed.

More recently, ultrafiltration has been used the for isolation of plasmid DNA. Hirasaki et al., J. Membr. Sci., 106: 123-129 (1995). The starting concentration of plasmid DNA is much greater than that of native nucleic acid molecules present in urine.

A need therefore remains for a method for accurately and efficiently processing nucleic acids and specific target nucleic acids using a concentration technique that is applicable for automation without detriment to workflow.

### SUMMARY OF THE DISCLOSURE

The disclosure relates to the concentration of nucleic acid molecules in a urine sample. The sample may be from any animal or subject that produces urine. In many cases, the urine sample is from a human subject, such as a human patient under clinical care or evaluation.

In general because nucleic acids are present in urine at very low concentrations, in order to obtain a sample having sufficient quantity of nucleic acid for subsequent detection by molecular techniques, large volumes of starting material (urine) from an individual is needed. For certain categories of target nucleic acids that are present at even lower levels relative to total urine nucleic acid, (e.g. cell-free or circulating DNA), the processing of a requisite larger volume of starting sample has been cumbersome. Previously, samples of 20 ml or greater starting volume required dividing the sample into several aliquots to then be processed in parallel.

The disclosure provides methods for the concentration of nucleic acids present in urine, the method comprising obtaining urine from a subject and removing water, cells, cell debris, peptides and salts from the urine by use of size-selective membrane and pressure; thereby obtaining a 10× to 20× or greater decrease in sample volume with a relative increase in concentration of nucleic acid molecules retained in the retentate.

In one embodiment, the method of concentration includes ultrafiltration with pressure.

The disclosed methods may thus be viewed as permitting the removal of water and other small molecules from a urine sample while selectively retaining nucleic acid molecules in the sample. The concentration of nucleic acid molecules in the sample would thus increase, while the concentration of the removed small molecules would remain relatively unchanged. The retained nucleic acid molecules may be double-stranded or singled-stranded, DNA or RNA, and complexed or free in solution. Complexed nucleic acid molecules include those that are in physical association with other molecules, such as other nucleic acid molecules, polypeptides, carbohydrates, or lipids or combinations thereof. Because the present method allows processing of a subject's urine as a single sample without limit on the starting sample's volume, the method may be automatized.

### BRIEF DESCRIPTION OF THE DRAWINGS

FIG. 1 shows a detection of Rnase P in a sample of urine (unconcentrated, concentrated, unconcentrated+clean up, or concentrated+clean up). “C-U” refers to concentrated urine; “C-Ex-U refers to concentrated urine that has also been subjected to SAX extraction (2M salt), P-6 clean up; “U/C-Ur” refers to unconcentrated urine; and “U/C-ex-U” refers to unconcentrated urine that has also been subjected to SAX extraction (2M salt), P-6 clean up.

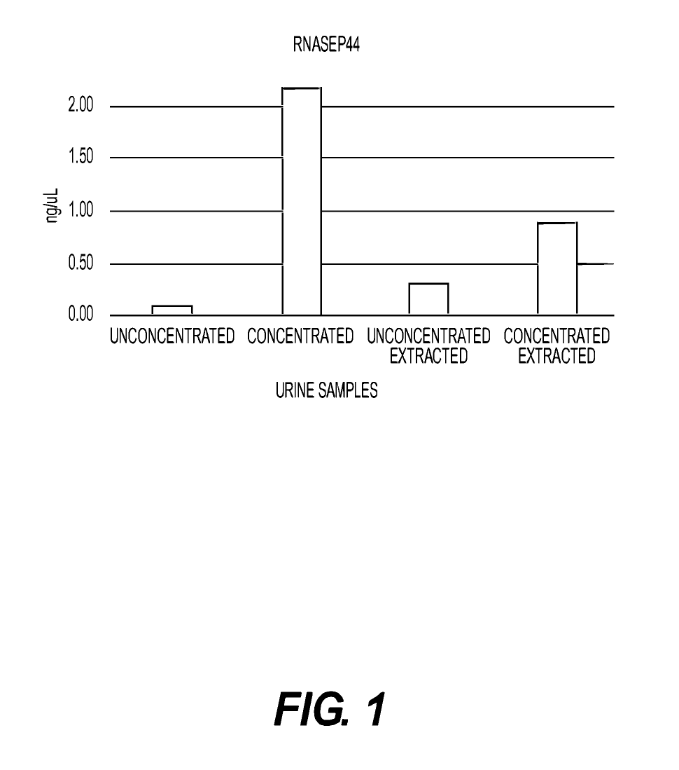

### DETAILED DESCRIPTION OF THE INVENTION

Ultrafiltration with centrifugation is the most often used method for concentrating plasmid DNA from larger volumes of fluid. “Separation of plasmid DNA isoforms using centrifugal ultrafiltration,” July 2012, Biotechniques. Plasmid DNA, however, differs substantially from nucleic acid molecules present in urine. Furthermore, it has been shown that the orientation of the membrane during centrifugation affects the quantity and quality of target molecules recovered. Beckwith et al., Sartorius Stedim Biotech, (“The Role of Ultrafiltration Membranes In The Recovery of DNA With Centrifugal Concentrators,” Intl Symposium on Human Identification, 2010) reported that centrifugal devices having horizontally-oriented membranes result in better retention of DNA and removal of inhibitory substances than concentrators with vertically-oriented membranes. Due to the inherently small concentration of target nucleic acids in urine, methods that result in reduced retention of nucleic acids is undesirable. Simply concentrating the nucleic acids by precipitation is inefficient with larger volumes of urine samples. Furthermore, safe and efficient method for removal of water, salts and other solutes from a larger volume urine sample, and a method which does not require centrifugation is desired.

The present method is ideal in that there is significantly less handling per sample, does not use hazardous reagents, and a large volume of urine can be processed as a single sample. The reduced processing time greatly augments workflow, rendering it particularly suitable for automation. The present method is particularly amenable to automation.

For certain target nucleic acids, a sample volume of 40 ml or greater is needed in order to obtain sufficient quantity of the target for subsequent detection by amplification or other molecular techniques. Depending on the hydration of the patient at the time of sample collection, up to 100 ml or greater sample volume may be needed. Using prior methods, a 100 ml sample of urine required its division into five separate aliquots of 20 ml each, thereby greatly increasing the handling and processing time and hindering workflow.

The present disclosure uses size-selective membranes with pore sizes that are smaller than the molecular weight of the target nucleic acid molecule(s) in conjunction with pressure to force filtration of water, salts, peptides and impurities through the membrane, while retaining desired nucleic acid molecules. The target nucleic acids may be concentrated 10×, 20×, 30×, 36× or greater in the retentate.

FIG. 2 is a diagrammatic representation comparing a prior method for purifying nucleic acids from a large volume of urine to a system according to the present method. In the prior method (A), a 100 ml sample is divided into five, 20 ml aliquots. The 5 aliquots are parallel processed by centrifugal ultrafiltration and then recombined into a single test sample. In the present method, the 100 ml sample is immediately processed by use of a device having a larger capacity fluid holder (20) coupled to a membrane (30). An inlet (40) of the holder (20) allows access to a pressure source. In operation, fluid and molecules smaller than the membrane's molecular weight cut-off pass through the membrane and into a downstream compartment (50). The system may be used if desired with negative pressure (vacuum). The system may also be used if desired with centrifugation.

The increase in nucleic acid molecule concentration provided by the disclosed methods is of at least 20-fold, which is readily understood by the non-limiting example of a urine sample of 80 milliliters (mL) that is reduced to 4 mL. Of course the same fold increase is seen with a reduction of 100 mL to 5 mL. At least a 25-fold or a 30-fold increase in concentration are also provided by the disclosed methods, such as by reducing 100 mL of urine to 4 mL or 90 mL to 3 mL, respectively.

The disclosed concentration methods utilize filtration through a size selective membrane that permits passage of water molecules and other small molecules based upon the molecular size cutoff of the membrane. In some embodiments, the molecular weight cutoff is 10,000 daltons or less, while in other embodiments, a molecular weight cutoff of 5,000 daltons or less is used. The use of a cutoff means that very small nucleic acid molecules, such as those smaller than the cutoff, will not be concentrated by the disclosed methods.

So in some cases, where only larger nucleic acid molecules are to be concentrated, a higher molecular weight cutoff (greater than 10,000 daltons) may be used. Non-limiting examples include cutoffs of 15,000 daltons, 20,000 daltons, 25,000 daltons, 30,000 daltons, 35,000 daltons, 40,000 daltons, 45,000 daltons, or 50,000 daltons or higher. The selection of cutoff size and the size of nucleic acid molecules may be made by the skilled person based on knowledge regarding the molecular weights of polynucleotides and that for maximum retention (or recovery) the cutoff should be at least 50% smaller than the molecular size of the nucleic acid molecule of interest.

In some embodiments, the membrane is made of polyethersulfone (PES) with a molecular weight cutoff of 10,000 or 5,000 daltons. In other embodiments, the membrane is made of cellulose, such as regenerated cellulose or modified regenerated or cross-linked cellulose. Cellulose triacetate membranes, cellulose composite membranes and microporous membranes are also suitable for use in the methods described herein. Cellulose has some desirable properties, such as hydrophilicity, low non-specific binding, and low fouling characteristics. In some embodiments, regenerated cellulose hollow fibers, flat sheet polyvinulidene fluoride (PVDF) and PES membranes are suitable. Suppliers include, for example, manufacturers such as, for example, Vivaproducts, Asahi, Millipore, Pall, Sartorius, Sartocon, GE Healthcare Biosciences AB, may also be used. In some embodiments, the membrane may be a polyethersulfone (PES) membrane. In some embodiments, the membrane may be a modified regenerated cellulose such as, for example, HYDROSART® membrane. In other embodiments, the membrane may be a Ultracel® low binding. In some embodiments, the membrane may be a Regen membrane. The ultrafiltration membrane used may be of cellulose or regenerated cellulose. Cellulose ester membranes can be composed of cellulose monoacetate, cellulose diacetate, cellulose triacetate, cellulose propionate, cellulose butyrate and cellulose acetobutyrate or other suitable cellulose esters, or cellulose nitrate, methylcellulose or ethylcellulose, and also mixtures thereof, preference being given to cellulose acetates, more particularly cellulose diacetate.

Pretreatment of the membrane is not necessary, but may be performed if desired depending on the Skilled Artisan's particular application. The membrane does not require pre-wetting.

A variety of membranes are available commercially and selection may be based on factors such as, for example, retention of the target nucleic acid sequence, retention consistency; low protein binding, overall process economics; scalability; mechanical robustness; and/or ease of use. The pore size of ultrafiltration membranes is generally defined by specifying the limit at which 50% 80%, 90%, or 95% of the molecules of at least a particular molar mass are retained (molecular weight cutoff, MWCO).

Selectivity of a membrane is understood to mean its ability to distinguish between the components of a mixture.

Selection of a membrane having a suitable pore size may be determined empirically such as, for example, via a spiking study, thermal and hydraulic stress resistances; and will include consideration of the user's particular application. Generally, a suitable membrane is one that has of about half, or about one third to one fifth of the desired or target nucleic acid molecular weight. For example, a membrane rated at 4 kDa-10 kDa is useful for retention of nucleic acid sequences having about 15 bp to about 30 bp or greater, or about 2 kDa to about 5 kDa MW or greater. A membrane rated at about 50 kDa is suitable for retention of double-stranded nucleotides of about 300 bp or greater. A membrane rated at about 100 kDa is suitable for retention of nucleic acids of about 600 bp or greater. A membrane rated at about 125 kDa MWCO is suitable for retention of nucleic acids having about 650 bp or greater or about 900 bp or greater, depending on the amount of pressure or vacuum applied.

The disclosed methods may be used to concentrate a urine sample of any starting volume. In some cases, the starting volume is 20 ml or more, 30 ml or more, 40 ml or more, 50 ml or more, 60 ml or more, 70 ml or more, 80 ml or more, 90 ml or more, or 100 ml or more. For these embodiments the membrane may have a surface area of at least 10 cm2 or more for contact with the urine sample. Of course the simultaneous use of more than one membrane to provide the total surface area may be optionally used. The surface area determines, in part, the available surface for non-specific binding and fouling. For use with larger urine volumes, the membrane surface area may be increased accordingly. Non-limiting examples include surface areas of at least 5 cm², at least 10 cm², at least 20 cm², at least 24 cm², at least 26 cm², at least 28 cm², at least 30 cm², at least 35 cm², at least 40 cm², at least 45 cm², or at least 50 cm² or more. For concentration of particular target nucleic acids that are generally present in urine at low concentrations (e.g. cell-free DNA/RNA, circulating cell-free DNA/RNA) a smaller membrane surface area is preferred. Because nonspecific binding is proportional to membrane area, a smaller membrane area aids in reducing nucleic acid loss and aids in increasing recovery of target nucleic acids.

The disclosed methods may be performed with centrifugation as the force for flow of urine through a membrane. Because of differences in urine samples from different subjects, the rate of flow is not identical for all samples. In some cases, urine is observed to be less clear, or visibly cloudy, which may slow its rate of flow. It has been observed that urine that has been previously frozen, including urine subjected to long-term storage at 4° C. or less will contain precipitates upon thawing. The present methods are as effective at concentrating previously frozen and/or stored urine as with fresh urine samples. Little or no reduction in concentration efficacy and quality of recovered nucleic acids has been observed with the present method.

So in some embodiments, the force may be positive pressure applied on the urine sample to increase the rate of passage (filtering) through the membrane. The pressure to apply may be readily determined by the skilled person based on the membrane type, membrane thickness, supporting structure for the membrane, and other relevant criteria. In some cases, the positive pressure is 5 bar (75 psi) or less. In some cases, the positive pressure is about 0 to 5 bar (approximately about 0 to 70 psi). Non-limiting examples include 4.5 bar, 4.0 bar, 3.5 bar, or 3.0 bar or less. As recognized by the skilled person, the greater the force, the higher the rate of filtration.

In other embodiments, the force may be a negative force applied below the membrane to draw the urine through. In some cases, this is readily accomplished by applying a vacuum below the membrane. The negative pressure may be determined in a manner analogous to positive pressure as described above. And similar examples of pressure may be used.

In other embodiments, the force may be a centrifugal force on the urine sample to increase its passage rate through a membrane. Again, the force to apply may be readily determined by the skilled person based on the membrane type, membrane thickness, supporting structure for the membrane, and other relevant criteria. In some cases, the force is 2000 g or less.

Concentration of nucleic acids may be performed at any suitable temperature for the samples, membranes, and devices used. In some cases, room temperature is used. In other cases, a reduced temperature below room temperature, such as 4° C., may be used.

The method of the invention can also be combined with other methods, resulting in a substantial increase in purity of nucleic acid sample. For example, the retentate may be further processed with a silica clean-up method, anion-exchange membrane, ethanol precipitation or processed with commercially available kits such as, for example, Qiagen's QiaQuick column for purification and/or isolation of sample nucleic acids. or Promega P-6 column. Target nucleic acids may then be detected using diagnostic assays such as ddPCR, fluorescence ddPCR, Real-Time PCR, fluorescence Real-Time PCR, RNA amplification, or other methods known to the skilled artisan.

The method is particularly suitable for automation. Manual processing of biofluid samples involves a great deal of repetitive handling steps. This is not only potentially hazardous, it is time-consuming and tedious and subject to human error. Such errors could result in quantitation, diagnostic or target detection errors.

Having now generally provided the disclosure, the same will be more readily understood through reference to the following examples which are provided by way of illustration, and are not intended to be limiting of the disclosure, unless specified.

### EXAMPLES

### Example 1

### Urine Sample

Urine samples of about 20 ml, 40 ml, 60 ml, 80 ml, 100 ml , 500 ml or greater, from human subjects were collected and stored at 4° C. Optionally, samples can be stabilized with EDTA and placed in long-term storage at −80° C.

### Example 2

### Concentration of Urine

Cellulose, Regenerated cellulose, or PES membranes with a total surface area of 23.5 cm² and a 5000 Dalton cutoff was used in a concentrator compartment with a 100 mL capacity. The compartment was attached to a filtrate container below, where fluid must pass through the membrane to enter the filtrate container. The compartment was fitted with a pressure head and seal to permit application of positive pressure to a urine sample.

A volume of 40 to 90 mLs of urine was placed in the compartment. The compartment was sealed and pressure applied up to about 5 bars with nitrogen gas. The pressurizing gas may optionally be disconnected.

Concentration was performed until the level of concentrated urine was reduced to about 3 to 4 mLs. This generally takes about 1 to 3 hours, but can be performed for a shorter amount of time or can be performed a longer amount of time depending on the final volume desired and/or characteristics of the urine sample. After concentrating, the seal was disrupted to release any residual pressure. The concentrated urine was withdrawn from the compartment and place into a labeled 15 mL tube. Residual liquid may be removed with a P200 pipette and added to the same tube. The vessel may be rinsed with a small amount of fluid (e.g. suitable buffer) to obtain nucleic acids non-specifically bound to the membrane and/or vessel. The wash fluid may be removed and combined with the concentrated urine in the tube. Optionally, this rinse procedure may be repeated and the wash fluid added to the tube.

The material in the tube contains nucleic acid molecules from the original urine sample in concentrated form. The nucleic acids may be isolated or extracted by methods known to the skilled person. Non-limiting examples include binding to, and elution from, an anion exchange medium or use of commercially available gel or chromatography columns or beads or magnetic beads.

### Example 3

A 100 ml sample of urine from a healthy, normal donor was transferred from the urine collection cup to a Vivacell device. The sample chamber was sealed and pressure at about 3 bar was applied for about 3 hours. The pressure was released and the retentate (about 3 ml) transferred to a tube. The Vivacell vessel & membrane was washed with 0.5 ml Binding Buffer (100 mM Tris, 50 mM EDTA, 0.2% Tween) to remove non-specifically bound nucleic acids adhered to the membrane and vessel. The wash fluid was added to the retentate resulting in a final sample volume of 7 ml. An aliquot of this concentrated urine was then subjected to a strong anion exchange extraction (“SAX”) and cleaned up with a polyacrylamide gel column to remove salts (Promega P-6, Bio-Rad, USA)

### Example 4

A 90 ml sample of urine from a healthy, normal subject was transferred to a device including a regenerated cellulose membrane. The device was sealed and subjected to centrifugation at 4000 rpm for 70 minutes. The retentate, having a volume of 2.5 ml was transferred to a separate tube. The device and membrane was washed with 500 μl of buffer (100 mM Tris, 50 mM EDTA, 0.2% Tween-20).

### Example 5

Detection of Rnase P copy number was performed on urine sampled from a normal human subject using Droplet Digital Polymerase Chain Reaction (ddPCR) with the QX100 system (Bio-Rad, USA). ddPCR measures absolute quantities by counting nucleic acid molecules encapsulated in discrete, volumetrically defined, water-in-oil droplet partitions. Hindson et al., High-Throughput Droplet Digital PCR System for Absolute Quantitation of DNA Copy Number, Anal. Chem. 2011, Nov. 15; 83(22):8604-8610.

Quantification of amplifiable DNA (RNaseP) detectable in urine processed by the present concentration method (30×) was compared to results obtained with concentrated urine (30×) which had been additionally subjected to size exclusion chromatography (Micro Bio-Spin with Bio-Gel P-6, BioRad, USA) to remove salts and impurities.

### Urine Samples (Concentrated or Unconcentrated), DNA Extraction and Preparation

The same source urine was processed by the concentration method as described in Example 2 and then used directly for ddPCR (“C-U”), or further processed by SAX extraction (2M salt) P-6 column clean up (Bio-Rad) to remove salts and molecules smaller than 6 kDa (“C-Ex-U”).

Urine from a normal human subject was obtained and stored at 4° C. before and after processing. For concentration, 720 ml of the urine was processed to 20 ml by the present method. The membrane was washed with 4 ml of wash buffer which was then added to the concentrate, bringing the final volume to 24 ml (30× concentration). The same source urine was extracted with SAX magnetic beads followed by a polyacrylamide column cleanup (Bio-Rad P-6 minicolumn).

A pool of male healthy donor DNA (“Promega XY”) was used as a positive control and standard for detectable DNA in the assay.

Below are the sample dilutions tested:

| Unconcentrated urine (U/C Ur) | 1x |
|---|---|
| Unconcentrated urine (U/C Ur) | 1/10 |
| Concentrated urine 36x concentrated (C U) | 1x |
| Concentrated urine 36x concentrated (C U) | 1/10 |
| Concentrated urine 36x concentrated (C U) | 1/50 |
| Unconcentrated extracted urine (U/C Ex U) | 0.41 ng/μL; 1x |
| Unconcentrated extracted urine (U/C Ex U) | 1/10 |
| Concentrated extracted urine (C Ex U) | 6 ng/μL; 1x |
| Concentrated extracted urine (C Ex U) | 1/10 |
| Concentrated extracted urine (C Ex U) | 1/50 |

ddPCR Quantitation of RnaseP DNA

The samples were quantified for copy number of the RnaseP gene using the cycling parameters below. The assay allows for the precise quantitation of RnaseP DNA down to 1 copy of an RnaseP standard. Briefly, 1 μl of urine (unconcentrated, concentrated or concentrated+extraction) or DNA standard (1 ng or 10 ng) was added to 20 μl of Master Mix (Table 1) and amplified using the following cycling program:

PCR program

| | | |
|---|---|---|
| 95° C. | 10 min | |
| 98° C. | 15 sec | |
| 60.5° C. | 1 min | x40 |
| 12° C. | hold | |

TABLE 1
Master Mix

| 20 μl reaction = 22 μl setup | Final | | |
|---|---|---|---|
| [vendor] | Concentration | | μl |
| Nuclease free H20 [IDT] | n/a | | 8.4 |
| 2x ddPCR Supermix/probe [BioRad] | 1x | | 11.0 |
| Uracil-DNA Glycosylase 1 U/μl (UDG) [NEB] | 0.02 | u | 0.44 |
| 0.1M MgCl2 [AppliChem] | 4 | mM | 0.88 |
| Forward primer RP-T-F 100 μM [EGT] | 400 | nM | 0.0898 |
| Reverse Primer RP44bp_R 100 μM [IDT] | 400 | nM | 0.088 |

| | | |
|---|---|---|
| Urine (concentrated or concentrated + P-6) | n/a | 1.100 |

22.0 μl

TABLE 2

| | |
|---|---|
| RP-T-F | 5′-CACTGGGAGGGAAGCTCATCAG(HEXdT)G-3′ |
| RP122 bp-R | 5′-CGAAGCTCAGGGAGAGCC-3′ |
| RP44 bp-R | 5′-ACAGGACGCACTCAGCTC-3′ |

### RESULTS

Detectable DNA concentration as determined by ddPCR are shown in Table 3. Expected copy numbers for control, DNA standard measured by ddPCR (Promega XY 1.32 ng or 11.32 ng) were comparable to input standard values (Promega XY 1 ng or 10 ng). The average total copy number detected in a 1 μl sample of concentrated urine (“Cone”) was about 663 as compared to copy number of about 272 for 1 μl of concentrated urine that was further extracted (“Conc Ext”), about 32 copy number for 1 μl of unconcentrated urine (U/C), and about 93 copy number for 1 μl of unconcentrated+extracted urine (“U/C Ext”). Urine concentrated by the present method resulted in a greater than 10-fold, or greater than 20-fold or about a 21-fold greater level of sensitivity for detection of nucleic acids present in a urine sample.

As shown in FIG. 2, amplifiable DNA was successfully detected in concentrated urine without the need for subsequent extraction or processing and at a level of sensitivity comparable to purified DNA standard obtained from a pool of male healthy donors. Total RnaseP detected in concentrated urine (about 2.19 ng/μl) was about 2.4-fold greater than that detected in concentrated+extracted urine (about 0.9 ng/μl), about 20-fold greater than that detected in unconcentrated urine (0.11 ng/μl), and about 7.1-fold greater than that detected in unconcentrated+extracted urine (0.31 ng/μl).

TABLE 3

| | | Average | |
|---|---|---|---|
| Sample | total copies | total copies | ng/μl |
| U/C Urine 1x | 28 | 32 | 0.11 |
| U/C Urine 1x | 36 | | |
| U/C Urine 1/10 | 2 | 5 | 0.02 |
| U/C Urine 1/10 | 7 | | |
| Conc Urine 1x | 610 | 663 | 2.19 |
| Conc Urine 1x | 716 | | |
| Conc Urine 1/10 | 86 | 84 | 0.28 |
| Conc Urine 1/10 | 82 | | |
| Conc Urine 1/50 | 18 | 14 | 0.05 |
| Conc Urine 1/50 | 10 | | |
| U/C Ext Urine 1x | 84 | 93 | 0.31 |
| U/C Ext Urine 1x | 102 | | |
| U/C Ext Urine 1/10 | 8 | 6 | 0.02 |
| U/C Ext Urine 1/10 | 4 | | |
| Conc Ext Urine 1x | 274 | 272 | 0.90 |
| Conc Ext Urine 1x | 270 | | |
| Conc Ext Urine 1/10 | 24 | 22 | 0.07 |
| Conc Ext Urine 1/10 | 20 | | |
| Conc Ext Urine 1/50 | 4 | 7 | 0.02 |
| Conc Ext Urine 1/50 | 9 | | |
| Promega XY 1 ng/μL | 394 | 399 | 1.32 |
| Promega XY 1 ng/μL | 404 | | |
| Promega XY 10 ng/μL | 3460 | 3430 | 11.32 |
| Promega XY 10 ng/μL | 3400 | | |

TABLE 4

| Sample | Avg (ng/μl) |
|---|---|
| Unconcentrated | 0.11 |
| Concentrated | 2.19 |
| Unconcentrated Extracted | 0.31 |
| Concentrated Extracted | 0.90 |
| DNA standard (1 ng/μl) | 1.32 |
| DNA standard (10 ng/μl) | 11.32 |

### FIG. 2 Legend:

C-U Concentrated urine C
C-Ex-U Concentrated Urine C—SAX extracted 2M salt P-6 clean up
U/C-Ur Unconcentrated Urine C
U/C-Ex-U Unconcentrated Urine C—SAX extracted, 2M salt, P-6 clean-up

All references cited herein, including patents, patent applications, and publications, are hereby incorporated by reference in their entireties, whether previously specifically incorporated or not.

Having now fully described the inventive subject matter, it will be appreciated by those skilled in the art that the same can be performed within a wide range of equivalent parameters, concentrations, and conditions without departing from the spirit and scope of the disclosure and without undue experimentation.

While this disclosure has been described in connection with specific embodiments thereof, it will be understood that it is capable of further modifications. This application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth.

What is claimed is:

1. A method of concentrating nucleic acid molecules in a urine sample from a patient, wherein the nucleic acid molecules have a size the same or greater than a target nucleic acid molecule, the method comprising
a) obtaining the sample of urine from the patient, wherein the initial sample has an initial volume of about 20_ml or greater;
b) applying the sample to a membrane having a molecular weight cut-off that is about 20% to 50% smaller than a target nucleic acid molecule;
c) applying a pressure of about 0 to about 5 bar, or about 0 to about 70_psi to the sample in contact with the membrane; and
d) collecting a retentate, wherein the retentate comprises a 5-fold or greater concentration of a target nucleic acid as compared to its initial concentration in the urine.

2. (canceled)

3. The method of claim 1, wherein subsequent to concentration, the target nucleic acid has a 20-fold or greater concentration.

4. The method of claim 1, wherein the target nucleic acid has a 30-fold or greater concentration.

5. The method of claim 1, wherein the target nucleic acid has a 36-fold or greater concentration.

6-9. (canceled)

10. The method of claim 1, wherein the sample has an initial volume of about 80 ml or greater.

11. The method of claim 1, wherein the sample has an initial volume of about 200_ml or greater.

12. The method of claim 1, wherein said membrane is made of cellulose, regenerated cellulose or polyethersulfone (PES) having a molecular weight cutoff of about 10,000 daltons or less.

13. The method of claim 1, wherein the molecular weight cutoff is about 5,000 daltons or less.

14. The method of claim 1, wherein said membrane has a surface area of about 20 cm² or more in contact with the urine sample.

15. The method of claim 1, wherein said concentrating comprises applying positive pressure on the urine sample to increase the rate of flow through the membrane.

16. The method of claim 15, wherein said positive pressure is about 5 bar (about 75 psi) or less.

17. The method of claim 1, wherein said concentrating comprises applying a vacuum below the membrane to increase the rate of flow through the membrane.

18. The method of claim 1, wherein the method is automated.

19. (canceled)

20. The method of any one of claims 1-19 wherein the concentrated urine is used in a diagnostic assay.

21. (canceled)

22. A system for automatically concentrating nucleic acids in urine comprising:
a size-selective membrane housed or coupled to a fluid container means,
an inlet interface coupled to a means for providing gas pressure, and
a controller for automated control of dispensing urine into the fluid container means and operation of application and release of the gas pressure.

23. (canceled)

24. The method of claim 1, wherein the sample has an initial volume of about 90 ml or more.

25. The method of claim 1, wherein the sample has an initial volume of about 100 ml or more.

25. The method of claim 1, wherein the retentate has a volume of 4 ml or less.

26. The method of claim 1, wherein the retentate has a volume of 3 ml or less.


SEQUENCE LISTING (3 sequences)

SEQ ID NO: 1 - DNA, 24 bp, Artificial Sequence
  Description of Artificial Sequence Synthetic oligonucleotide
cactgggagggaagctcatcagtg

SEQ ID NO: 2 - DNA, 18 bp, Artificial Sequence
  Description of Artificial Sequence Synthetic oligonucleotide
cgaagctcagggagagcc

SEQ ID NO: 3 - DNA, 18 bp, Artificial Sequence
  Description of Artificial Sequence Synthetic oligonucleotide
acaggacgcactcagctc